(19) United States
(12) Patent Application Publication
(10) Pub. No.: US 2022/0265825 A1
(43) Pub. Date: Aug. 25, 2022

(54) OPTHALMIC COMPOSITION OF BEVACIZUMAB

(71) Applicant: GENNOVA BIOPHARMACEUTICALS LIMITED, Pune, Maharashtra (IN)

(72) Inventors: Kushal GARIMA, Pune, Maharashtra (IN); Swarnendu KAVIRAJ, Pune, Maharashtra (IN); Arjun RAGHUWANSHI, Pune, Maharashtra (IN); Sanjay SINGH, Pune, Maharashtra (IN)

(73) Assignee: GENNOVA BIOPHARMACEUTICALS LIMITED, Pune, Maharashtra (IN)

(21) Appl. No.: 17/631,863

(22) PCT Filed: Jul. 31, 2020
(86) PCT No.: PCT/IN2020/050677

§ 371 Date: Jan. 31, 2022

(30) Foreign Application Priority Data
Aug. 1, 2019 (IN) 201921017385

(51) Int. Cl. A61K 39/395; C07K 16/22; A61K 9/00; C07K 1/36; A61K 47/26; A61K 47/02; A61M 5/00

(52) U.S. Cl. CPC A61K 39/39591; C07K 16/22; A61K 9/0048; C07K 1/36; A61K 47/26; A61K 47/02; A61M 5/002; C07K 2317/14; C07K 2317/24

(57) ABSTRACT

The present invention pertains to an ophthalmic composition of Bevacizumab, in a single use prefilled syringe which is safe, non-toxic and efficacious for administration during the shelf life, characterised in that the ophthalmic solution is controlled with respect to the number of sub-visible particulate matter with significantly reduced endotoxin levels and with low aggregate level that are suitable for intravitreal use during the shelf life of 2 years of the product.

### CROSS-REFERENCE TO RELATED APPLICATIONS

The present application claims priority under 37 U.S.C. § 371 to International Patent Application No. PCT/IN2020/050677, filed Jul. 31, 2020, which claims priority to and the benefit of Indian Patent Application No. 201921017385, filed on Aug. 1, 2019. The contents of these applications are hereby incorporated by reference in their entireties.

### FIELD OF THE INVENTION

The present invention relates to the field of biotechnology and drug delivery. In particular, the present invention relates to an ophthalmic composition of bevacizumab and a device comprising the said composition.

### BACKGROUND OF THE INVENTION

Bevacizumab is a recombinant humanized monoclonal antibody that contains human framework regions and the complementarity-determining regions of a murine antibody that binds to and inhibits the biologic activity of human vascular endothelial growth factor (VEGF). Bevacizumab is licensed to treat various cancers, including metastatic colorectal cancer (mCRC), Non-Squamous Non-Small Cell Lung Cancer (NSCLC), Metastatic Breast Cancer (MBC), Metastatic Renal Cell Carcinoma (mRCC), glioblastoma and late-stage cervical cancer. In these indications, bevacizumab has proved its efficacy in terms of patient's safety and survival. In addition, it is also being used as an off-label drug to treat wet age-related macular degeneration (wAMD), choroidal neovascularisation (CNV), diabetic macula edema (DME), retinal vein occlusion (RVO) and neovascular glaucoma (NVG). Alternatively, another monoclonal antibody, Ranibizumab, an USFDA approved drug for wAMD, CNV, DME etc. is also in the market. Though there appears to be no difference in the efficacy and safety, between bevacizumab and ranibizumab proved by several studies, a huge cost advantage exists if bevacizumab is used instead of ranibizumab.

As bevacizumab is approved for cancer and it is available in 100-mg or 400-mg vials meant to provide the complete dose for an individual according to the body weight. When using Bevacizumab for wAMD, ophthalmologists typically inject a much smaller amount, as low as 1.25 mg per dose intravitreally probably repeatedly from the small vial (multi-dose usage). Any drug meant for intravitreal administration needs to be prepared and maintained in a sterile environment. There is a risk of contamination when the injection would be prepared in the vial by drawing of the reconstituted solution in the device before administration. A contaminated drug puts the patient at risk of several further infections such as endophthalmitis, alpha hemolytic streptococcus infection. In some parts of globe, doctors themselves aspirate the needed drug from the original vial and the same vial is used for many consecutive patients which have been proven a wrong practice in context to patient's safety because increasing number of punctures from the same vial enhances the chances of contamination and infection to the patients. Further, there may be inaccuracies of dose due to the human errors by the administering medical or paramedical personnel.

In some countries, compounding pharmacies repackage multiple syringes from the original vial. The need to repackage the drug from the available size vial into a smaller dose enhances the probability of transmission of contamination, especially since it is difficult to maintain aseptic conditions during such procedures and there is no manner to check whether such procedures are being followed at the compounding pharmacies. FDA has documented and issued warning notices at several instances regarding the use of aseptic techniques and dosage control of these compounding pharmacies, but such practices are still rampant. In addition, several compounding pharmacies have recalled bevacizumab repackaged syringes due to sterility concerns and U.S. FDA is now warning the medical and paramedical team against the utilization of repackaged injections of bevacizumab for intraocular use. In addition to that, bevacizumab is manufactured for intravenous administration and hence the criteria for particle size distribution is critical. If the particle is not controlled, it will create irritation and/or inflammation in a sensitive organ such as eye. It has been reported for such repackaged injectable ophthalmic solutions that particle size distribution falls outside the limits set by US Pharmacopeia and generally not standardized and uniform across different compounding pharmacies.

In addition to safety issues, the drug itself may have variable efficacy associated with product aliquot, handling, and distribution. Deterioration may also be subject to the particular packaging used, and reduced stability of repackaged bevacizumab may be associated with duration of storage. In addition to the potential safety implications of these changes, the efficacy of the drug may also be affected. Also, storage stability of repackaged bevacizumab is also a main concern and the guidelines issued by Indian DCGI regarding repackaging of bevacizumab, it has been recommended to use repackaged bevacizumab syringes not more than 14 days in specified condition, in order to prevent to degradation and denaturation. But there is no effective mechanism to ascertain to examine the time limit after reconstitution and/or denaturation or degradation in the storage period. Further reconstitution of packaged product for administration is a complex and cumbersome procedure and may not always result in a homogenous solution. Moreover, there are reports in which counterfeited Avastin is additionally traced internationally which have turned out to be increasingly challenging, as global supply chains have become more complex.

Certain issues relating to compounding of bevacizumab into small units for intravitreal use and counterfeited bevacizumab may be reduced by implementation of strict regulatory guidelines but the problems of prior art like Bacterial endotoxin Limit (BET) and Particulate matter test (PMT) limit need specific technical solutions and manufacturing control to eliminate the problems of prior art and to provide a product acceptable for ophthalmic intravitreal injections.

There are increasing reports of adverse events associated with intravitreal injection of bevacizumab, including incidences of intraocular sterile inflammation, infectious endophthalmitis, and elevated intraocular pressure. Certain reported adverse events associated with the intravitreal administration of bevacizumab may be related to the way it is manufactured, or to deterioration in the quality of the drug because of the repackaging into plastic syringes. In addition to that, silicone droplets that comes from the needles and syringes used for the intravitreal injections makes the condition worst.

There are issues relating to product quality of the prior art products as below:
Bevacizumab currently only meets intravenous quality standards, rather than the more stringent ophthalmic standards regarding sub-visible particle matter and endotoxin levels.
The presence of sub-visible particles and/or silicon oil is critical in the case of intravitreal injection (accumulation in the eye, leading to potential for severe intraocular inflammation).
No data is presented to consider the quality of the product after repackaging from a microbiological as well as a physiochemical point of view. Nor is the compatibility of the solution with the primary packaging considered.
There has been no examination of the shelf-life setting of repackaged bevacizumab, and the impact on safety and quality in most part of the globe.

Hence, there is an urgent need for ophthalmic composition for Bevacizumab which is safe, non-toxic, and efficacious for single use administration with a long shelf life.

### OBJECT OF THE INVENTION

An object of the invention is to provide an ophthalmic composition of Bevacizumab with controlled particulate matter, bacterial endotoxin, aggregate during the shelf life, a process for preparing the composition and a device for administration of the composition which is safe, non-toxic and efficacious for single use administration.

### SUMMARY OF THE INVENTION

The present invention discloses an ophthalmic composition of Bevacizumab, which is administered as a single use pre-filled syringe. The Ophthalmic composition of the present invention controls the limit of Bacterial Endotoxin Test (BET), particulate matter and the aggregates during the period of its shelf-life of 2 years. The present invention also provides a process for obtaining the active ingredient, being bevacizumab with negligible or nil amount of Bacterial Endotoxin Test (BET) and particulate matter and there the composition of the present invention when formulated has very low level of aggregate, BET and particulate matter during the period of its shelf life and suitable for intravitreal use. The invention also provides a device such a prefilled syringe or kit, comprising the ophthalmic composition of bevacizumab.

### BRIEF DESCRIPTION OF FIGURES

FIG. 1 depicts the schematic representation of obtaining the ophthalmic composition of the present invention is controlled with respect to the number of sub-visible particulate matter, low endotoxin levels during the shelf life of the product.

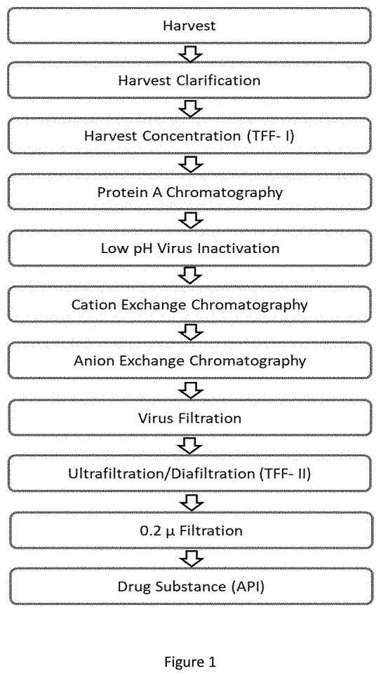

FIG. 2a and FIG. 2b depicts the sub-visible particle size of the sample of the present invention in comparison with that of the other samples in the prior art.

### DETAILED DESCRIPTION OF THE INVENTION

An ophthalmic composition of Bevacizumab which is safe, non-toxic and efficacious for administration during the shelf life, characterised in that the ophthalmic composition is controlled with respect to the number of sub-visible particulate matter, low endotoxin levels aggregates, during the shelf life of the product.

The ophthalmic composition of the present invention is preferably a solution. The ophthalmic solution may be present as individual injectable units of prefilled syringes.

The ophthalmic composition of the present invention is controlled with respect to Particulate matter. The particulate matter consists of mobile undissolved particles, derived from any component of the composition, impurities and any instability, other than gas bubbles, unintentionally present in the solutions, wherein the sub-visible particulate matter for intravitreal injections comprises;
(i) 1 to 50 particles ≥10 μm in diameter per ml
(ii) 0 to 5 particles ≥25 μm in diameter per ml, and
(iii) 0 to 2 particles ≥50 μm in diameter per mL

The presence of endotoxins in recombinant therapeutics is of major concern due to the diverse and potentially harmful biological activities of these molecules. Maintaining sterility in processes used in the manufacture of biologicals, together with stringent protocols for the preparation of equipment, helps to ensure that products have acceptably low levels of endotoxins. It is also evident from literatures that in anterior and posterior segment of eye, posterior segment (intravitreal injection site) is more sensitive to endotoxin and residence time of endotoxin in this segment is much more due to the higher viscosity and lower fluid flow in vitreous cavity which results in slow recovery of the vitreous cellular response. Even minimal endotoxin levels might result in the intraocular inflammation depending upon the sensitivity of the patients. Intravitreal injections of bevacizumab are administered every month and in a year 7-8 injections are required for wAMD. To avoid accumulation of endotoxin in eye by frequent intravitreal injections, it is obligatory to keep the endotoxin levels as low as possible. The outcome of sterile endophthalmitis and increase in intraocular pressure that developed after intraocular injection of bevacizumab have been reported at several instances. Although there are several theories about the aetiology of the intraocular inflammation, its causes are still unknown. Endotoxins which are normally occurring by products of commercially made immunoglobulins are supposed to play a major role in these sterile reactions.

Recently, there have been a few reports of TASS-like culture negative sterile endophthalmitis after IVB injections. This may be because of endotoxin related of breakdown products due to faulty storage. With the rising graph of number of IVB injections, the number of such cases are certain to rise, therefore necessitating the need to find a judicious way to deal with such situations. The aetiology of sterile endophthalmitis is poorly understood. However, there are numerous possible explanations. For instance, the common practice of dividing individual aliquots commercially available vials of bevacizumab, as a cost-effective method, may carry an increased risk of contamination with bacterial endotoxins. Bevacizumab compositions in prior art and as being administered contain traces of endotoxins at levels that are deemed safe for intravenous use, but which are still capable of inducing an inflammatory reaction intravitreally. In the post injection endophthalmitis extra drug manipulation that occurs during compounding also plays an important role. Concerns with respect to the repackaging of bevacizumab for intravitreal injection are increasing because of several outbreaks of infectious endophthalmitis reported in the United States.

The ophthalmic composition of the present invention controls the level of endotoxin concentration in the range of 0.001 to 0.4 EU/mg and preferably in the range of 0.001 to 0.2 EU/mg and more preferably in the range of 0.001 to 0.16 EU/mg.

The ophthalmic composition of the present invention may be aggregate free till the prescribed shelf life or may have an aggregate in the range 0.1 to 5%, more preferably 0.1 to 4% and most preferably 0.1 to 3.5% for a period of 2 to 3 years, preferably 2 to 2.5 years, most preferably 2 years.

The ophthalmic composition of the present invention can maintain the limits of Bacterial Endotoxin, particulate matter, and aggregate limits, during the period of shelf life, even if the cold chain is broken intermittently for a brief period, e.g. during transfer. In the said context, the product of the present invention has excellent tolerance in terms of stability and efficacy.

The ophthalmic composition of the present invention has no dose variation between the units as they can be prefilled to the determined dose through automation. Further the dose in an individual unit remains unchanged till the prescribed shelf life.

The ophthalmic composition of Bevacizumab of the present invention, comprises:
a. Bacterial Endotoxin Test (BET) in the range of 0.001 to 0.4 EU/mg and preferably in the range of 0.001 to 0.2 EU/mg and more preferably in the range of 0.001 to 0.16 EU/mg;
b. wherein the particulate matter is;
   i. 1 to 50 particles ≥10 μm in diameter per ml,
   ii. 0 to 5 particles ≥25 μm in diameter per ml, and,
   iii. 0 to2 particles ≥50 μm in diameter per mL; and;
c. wherein the composition has an aggregate of 0.1 to 5%, more preferably 0.1 to 4% and most preferably 0.1 to 3.5% for a period of 2 to 3 years, preferably 2 to 2.5 years, most preferably 2 years.

In another embodiment, there is provided a process of preparing the composition controlled with respect to the number of sub-visible particulate matter, low endotoxin levels and is substantially or completely devoid of silicon oil during the shelf life of the product of the present invention comprising the steps of:
i. culturing the cells of bevacizumab by continuous fermentation method in CHO cells;
ii. subjecting cell free harvest obtained from CHO cell culture to adsorption-based depth filtration for further clarification;
iii. concentrating the sample of step (ii) using single pass tangential flow filtration to obtain the concentrated harvest;
iv. subjecting the concentrated harvest of step (iii) to protein A chromatography to capture bevacizumab and obtaining an eluate containing partially purified bevacizumab;
v. subjecting eluate of step (iv) to low pH virus inactivation to obtain the viral inactivated sample;
vi. subjecting the viral inactivated sample of step (v) to a further cation exchange chromatography for additional purification to obtain an eluate containing primarily highly purified bevacizumab;
vii. subjecting the eluate of step (vi) to anion exchange chromatography to remove the trace impurities e.g. Host Cell Proteins (HCP), Host Cell DNA (HCD), endotoxins;
viii. subjecting the eluate of step (vii) to virus reduction filtration and complete removal of viruses;
ix. concentrating and diafiltrating of the sample of step (viii) to obtain bevacizumab; and;
x. formulating of the sample obtained from step (ix) to achieve the composition of bevacizumab of the present invention.

The low virus inactivation may be carried out at the pH in the range of pH 3.0 to 5.0 preferably in the range of 3.0 to 4.0, most preferably in the range of pH 3.5 to 4.0.

The cation exchange chromatography of the eluate may be conducted by using a matrix or stationary phase selected from the group comprising SO₃⁻Sulfoisobutyl, SO₃⁻Sulfoethyl, Sulfopropyl, Carboxymethyl, etc., preferably the chromatography matrix or stationary phase used is Sulfonate.

The anion exchange chromatography of the eluate may be conducted by using a matrix or stationary phase selected from the group comprising Diethylaminoethyl, Quarternaryamine,Polyquaternium, N-benzyl-N-methyl ethanol amine etc., preferably the chromatography matrix or stationary phase used is Quarternaryamine.

The diafiltration may be conducted in the presence of TFF-II Diafiltration Buffer.

The process as described herein results in a product and a composition with the desired endotoxin effect. For instance, the endotoxin level may be reduced sequentially as below: (See

Table A)

TABLE A
Depiction of Endotoxin level based on
the process of the present invention.

| Process steps | Endotoxin levels after each step |
|---|---|
| Single Pass Tangential Flow Filtration | NMT 10 EU/mg |
| (SPTFF) | |
| Protein A chromatography | NMT 10 EU/mg |
| Cation Exchange chromatography | NMT 5 EU/mg |
| Anion Exchange Chromatography | Less Than 0.5 EU/mg |
| At end of process | Less Than 0.16 EU/mg |

From Table A above, the process of the present invention results in several fold decrease in endotoxin content as determined by Gel Clot method. Before subjecting the harvested broth to the purification process the endotoxin content is very high. After Protein A affinity purification, the endotoxin content is not more than 10 EU/mg which is usually very high than the acceptable level. In addition, Anion exchange chromatography has been used to separate and remove endotoxin from the active being bevacizumab.

At the end of the anion exchange chromatography step, the endotoxin content in the protein is less than 0.5 EU/mg. Use of Anion exchange chromatography in process of the present invention enables the control the critical quality attributes of drug product e.g. BET, bioburden, aggregates and PMT in a narrower range.

Endotoxin removal is one of the most difficult steps in downstream process purification. To enable achievement of the endotoxin limits bevacizumab of the present invention, to obtain an ophthalmic composition having an endotoxin concentration of less than 0.4 EU/mg and preferably less than 0.2 EU/mg and more preferably less than 0.16 EU/mg. In addition, the process must involve:
(i) Use of sterile solutions and equipment to minimize the introduction of microorganisms,
(ii) Using filtration during purification with 0.22 micron filters,
(iii) Working at low temperature to minimize microbial growth,
(iv) Adding bacteriostatic agents to the purification,
(v) High grade raw materials,
(vi) Orthogonal purification steps,
(vii) Rigorous Cleaning in Place (CIP) procedures for vessels, columns and process equipment's.

The process of the present invention as disclosed herein is novel and results in a product with desired parameters. The process must be followed in the sequence as set out herein to achieve the product with desired properties. This process is evolved after much experimentation, trial, and error.

The process of the present invention provides an ophthalmic composition of Bevacizumab;
a. wherein the limits of Bacterial Endotoxin Test (BET) in the range of 0.001 to 0.4 EU/mg and preferably in the range of 0.001 to 0.2 EU/mg and more preferably in the range of 0.001 to 0.16 EU/mg;
b. wherein the particulate matter is;
   i. 1 to 50 particles ≥10 μm in diameter per ml,
   ii. 0 to 5 particles ≥25 μm in diameter per ml, and,
   iii. 0 to 2 particles ≥50 μm in diameter per mL; and;
c. wherein the composition has an aggregate of 0.1 to 5%, more preferably 0.1 to 4% and most preferably 0.1 to 3.5% for a period of 2 to 3 years, preferably 2 to2.5 years, most preferably 2 years.

In another embodiment, the present invention discloses an ophthalmic composition comprising, bevacizumab, a buffer, a stabilizer and a surfactant.

The concentration of bevacizumab in the composition may be in the range of 24 mg/ml to 26 mg/ml, preferably in the range of 25 mg/ml to 26 mg/ml, more preferably 25 mg/ml.

The buffer present in the composition may be selected from the group comprising phosphate, citrate, acetate, histidine, succinate, gluconate, glycine and the like, more preferably phosphate buffer. The concentration of buffer may preferably be in the range of 40 mM to 60 mM, more preferably in the range of 50 mM to 60 mM. The pH of the buffer may be preferably in the range of 6.0 to 7.0, more preferably 6.1 to 6.3.

The stabilizer may be a saccharide. The saccharide may be a monosaccharide, disaccharide, trisaccharide, polysaccharide, sugar alcohol, reducing sugar, nonreducing sugar, etc. Examples of saccharide herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose and the like. The preferred saccharide may be a non-reducing disaccharide, such as trehalose.

The amount of saccharide in the composition may be in the range of 40 mg/mL to 70 mg/mL, preferably the range may be 45 mg/mL to 65 mg/mL, more preferably the range is 50 mg/mL to 60 mg/mL.

The surfactant present in the composition refers to a surface-active agent, preferably a nonionic surfactant. Examples of surfactants herein include polysorbate (for example, polysorbate 20 and, polysorbate 80). The preferred surfactant herein is polysorbate 20.

The amount of surfactant in the composition may be in the range of 0.2 mg/mL to 0.6 mg/mL, preferably in the range of 0.3 mg/mL to 0.5 mg/mL, more preferably in the range of 0.35 mg/mL to 0.45 mg/mL.

The composition of the present invention is a homogenous solution and stable solution during the shelf life of the product and is therefore a synergistic composition.

In yet another embodiment, the ophthalmic composition of the present invention may be administered as predetermined dose be in the form of a vial, a cartridge or a pen or a prefilled syringe, preferably a pre-filled syringe, preferably single use pre-filled syringe, one with a fill volume of 140 microL to 200 microL and with a dose of 50 microL. The composition is substantially or completely devoid of silicon oil.

The body of the syringe may be composed of a material selected from the group comprising a polymer or glass, or indicia coated on any surface; a thermoplastic material; a polyolefin, such as cyclic olefin polymer, a cyclic olefin copolymer, or polypropylene; a polyester, such as polyethylene terephthalate; a polycarbonate; or any combination or copolymer thereof.

The plunger stopper of the pre-filled syringe may be composed of any suitable material selected from the group comprising chlorobutyl, bromobutyl or other halo alkyl rubbers. The plunger may optionally be coated with materials selected from the group comprising silicon, fluoropolymers, parylene and the like. Such a coating may be utilized to minimize the any interaction and/or friction that may be caused between different parts of the device or between the device and the drug during storage or during shipping. The tip of the plunger stopper may be flat or convex shaped. Plunger rings may be present for tight sealing of plunger and barrel wall. The stopper may be siliconized or coated with bromo, chloro, fluoro or related polymers.

The syringe may be optionally coated. An internal coating or layer, selected from the group comprising a tie coating, a barrier coating, a pH protective coating, or a lubricity coating, preferably lubricity coating with the lubricant selected from the group consisting of silicon oil, fluoropolymer, parylene and related compounds, in the range of 0.1 mg-0.4 mg of silicon-oil. The low siliconized borosilicate glass syringe barrels may be spray coated with silicon oil-in-water emulsion and subsequently may be heat-fixed (i.e. “baked silicone”) or Cross-linked. Such a coating and/or layering may be utilized to minimize the any interaction and/or friction that may be caused between different parts of the device or between the device and the drug during storage or during shipping. Preferably, the prefilled syringe may be completely free or devoid of silicone oil.

The predetermined dosage form of the present invention such as a prefilled syringe may be filled with an inert gas selected from the group comprising nitrogen, argon, helium, preferably nitrogen. The pre-filled pharmaceutical syringe may have a nominal maximum with a fill volume of 140 microL to 200 microL and with a dose of 50 microL.

The composition of the present invention may be administered as a pre-filled syringe. The composition of the present invention may be filled in pre-filled syringe for intravitreal injection. To decrease the likelihood of silicone oil droplets being injected into the eye, the prefilled syringe of the present invention may comprise a barrel which is substantially or completely free of silicone oil. The pre-filled syringe may comprise an internal coating of Silicon oil, such the internal coating may have 0.1 mg-0.4 mg of silicon-oil. In the alternate, the pre-filled syringe may be coated on the interior with a tie coating or layer, a barrier coating or layer, a pH protective coating or layer, and optionally a lubricity coating, preferably the coating may be fluoropolymer, parylene and related compounds. In one embodiment, the prefilled syringe may be completely free of silicone oil. The low siliconized borosilicate glass syringe barrels may be spray coated with silicon oil-in-water emulsion and subsequently may be heat-fixed (i.e. “baked silicone”) or Cross-linked. Such a coating and/or layering may be utilized to minimize interaction and/or friction that may be caused between different parts of the device or between the device and the drug during storage or during shipping.

In the present invention the interaction between the content and container is minimized and is critical for retaining the efficacy especially for maintaining the stability for long term storage/use for the entire period of shelf life being 2 years. The Applicant has undertaken several studies and made efforts such that this problem of the prior art is fully addressed in this regard.

In yet another embodiment, the ophthalmic composition of the present invention may be used to treat ocular disorders. The ocular disorder, may be selected from the group comprising wet Age related macular degeneration, choroidal neovascularization, retinal angiomatous proliferation, pathologic myopia, angioid streaks, Best disease, Adult vitelliform dystrophy, Central serous chorioretinopathy, Punctate inner choriodopathy, Multifocal choroiditis, Presumed ocular histoplasmosis syndrome, Choroidal osteoma, Toxoplasmosis, Uveitis, Pseudotumor cerebri, Peripapillary Idiopathic Retinal neovascularization, Proliferative diabetic retinopathy, Sickle cell retinopathy, Retinopathy of prematurity, Eales disease,

Macular edema, Diabetic retinopathy, Central retinal vein occlusion, Branch retinal vein occlusion, Pseudophakic, Uveitic, Occlusive vasculitis, Retinitis pigmentosa, Neovascular glaucoma, Central retinal vein occlusion, Branch retinal vein occlusion, Proliferative diabetic retinopathy, Central retinal artery occlusion, Ocular ischemic syndrome, Radiation induced, Radiation optic neuropathy, Radiation retinopathy, Breast cancer with choroidal metastasis, Melanoma associated neovascularization, Macroaneurysm, Vasoproliferative tumor, Coats disease, Juxtapapillary capillary hemangioma, Idiopathic macular telangiectasis, Polypoidal choroidal vasculopathy, Central serous chorioretinopathy, Nonarteritic anterior ischemic optic neuropathy, Herpetic corneal neovascularization, Cicatricial pemphigoid corneal neovascularization, Posterior capsular neovascularization, Corneal graft rejection neovascularization, Dry eye associated corneal neovascularization, Bleb revision, Adjunct to glaucoma filtering surgery, preferably wet age-related macular degeneration.

In yet another embodiment, the present invention provides a kit comprising the ophthalmic composition of the present invention in an administrable format, such as pre-filled syringe, vial or a cartridge, preferably a pre-filled syringe. The kit may comprise a pre-filled syringe, packed in a blister pack, which may itself be sterile on the inside. In one embodiment, syringes according to the invention may be placed inside such blister packs prior to undergoing sterilisation, for example terminal sterilisation. The blister pack may be formed of a suitable thermoplastic material such as a glass, polyolefin, a cyclic olefin polymer or a cyclic olefin copolymer, polypropylene or a polyester or any combination or copolymer thereof. The kit may also comprise a needle for administration of the ophthalmic composition of the present invention.

The needle may be any of 29 to 33 gauge×½ inch needle, though 31-gauge and 32-gauge, 33-gauge or 34-gauge needles could alternatively be used. The kit may also contain a product insert, which includes instructions for use.

The present invention discloses kit comprising the ophthalmic composition in a prefilled syringe, comprising a prefilled syringe, a needle with gauge in the range of 29 to 34 gauge, instructions for use in a blister pack.

The composition is stable at a temperature of 2-10 deg C, preferably 2 to 8 deg C at least for 2 years. The ophthalmic composition of the present invention is stable for at least two years and it not only maintains the safe ease of use but also have low sub visible particulate matter, BET and aggregate during its shelf life of two years.

### ADVANTAGES

The ophthalmic composition of the present invention has the following advantages, but the advantages may not be limited those listed herein below:
The product is highly stable as compared to other prior syringes compounded from the vial.
Comprises a BET within the limit as set out herein.
Contains a predetermined dosage, thereby reducing the dosage errors, and ensures disposal of the dosage form after a single administration, avoiding multiple use and thereby preventing multiple usage and infections stemming from multiple usage.
Has potentially less pre-injection and post-injection effects.
Has a shelf life of 2 years when compared with varied shelf life from 14 days to max 6 months (in India 14 days, FDA proposing it to limit only for 5 days after repackaging) of the prior art products.
The composition of the present invention having not more than 50 particles 10 μm in diameter per ml, not more than 5 particles ≥25 μm in diameter per ml, and not more than 2 particles ≥50 μm in diameter per mL.
The ophthalmic grade bevacizumab contained in the pre-filled syringe of the present invention, is stable at a temperature of 2 to 8° C. for the prescribed shelf life.
The composition of the present invention undergoes all prescribed quality checks on release unlike the re-packaged or compounded compositions of prior art.
Minimizes hospital visits, reduces drug waste, minimizes hospital and industrial wastes and eliminates risk of microbial contamination.

The present invention is illustrated by means of examples. The examples are meant for illustrative purposes and should not be construed as limiting.

### EXAMPLES

### Example 1: Process of Purification of Bevacizumab

The cell free harvest containing bevacizumab monoclonal antibody as obtained from perfusion technology-based bioreactors from CHO cells. The harvest was filtered with 0.2 μm filter and subjected to adsorption-based depth filtration for harvest clarification. Filtered harvest was concentrated to achieve a concentration of Bevacizumab by using single pass TFF module. The ‘Concentrated harvest’ obtained from TFF-I step was filtered using 0.2 μm filter and subjected to Affinity chromatography column packed with Mab Select Sure LX resin (GE Healthcare). The Column was equilibrated with equilibration buffer-1 (EB-1, Phosphate buffer: 20 mM, NaCl: 150 mM, pH 7.1±0.2) by passing 5 column volumes (CVs) of EB-1 at a flow rate of 150 cm/h. The sample was loaded onto the column with a linear flow rate of 150 cm/h followed by passing EB-1 of 5 CVs at a flow rate of about 150 cm/h. This was followed by 5 CVs of the wash buffer-1 (WB-1, Sodium Acetate: 40 mM, pH 6.0) at a flow rate of 150 cm/h. The bound target protein was eluted by passing about 5 CVs of elution buffer-1, (EUB-1, Sodium Acetate: 30 mM, pH 3.5) and collected based on the absorbance at 280 nm. The eluate (E-1) was analysed for protein quantity, Glycans, HCP, charge variants, aggregates and endotoxin content. At this step, the endotoxin content of E-1 is not more than (NMT) 10 EU/mg and aggregate content is NMT 5%. In next step, E-1 is subjected to low pH inactivation step where E-1 is incubated at room temperature with continuous mild stirring for 60 minutes at pH 3.7. After viral inactivation, the solution is diluted with CEX Equilibration buffer-2 (EB-2, Sodium Acetate: 50 mM, NaCl: 80 mM, pH 5.3) followed by filtration using 0.2 μm filter. In the next step, the cation exchange column (Capto S ImpAct, GE Healthcare) was equilibrated with 5 CVs of Equilibration Buffer-3 (EB-3, Sodium acetate: 50 mM, NaCl: 80 mM, pH 5.3) at a flow rate of 250 cm/h. The sample was loaded on to column at a flow rate of 250 cm/h which was followed by 3 CVs of the wash buffer-2 (WB-2, Sodium acetate: 50 mM, NaCl: 80 mM, pH 5.3) at the flow rate of 250 cm/h. Sample was run through a linear gradient of 0-15% B in 1.5 CV followed by 15-25% B in 20 CV using EB-3 and Elution buffer-2 (EUB-2, Sodium Acetate: 50 mM, NaCl: 500 mM, pH 5.3) at a flow rate of 250 cm/h and the fractions were collected based on UV 280. The eluate (E-2) was analysed for protein quantity, charge variants, aggregates and endotoxin content. After this step, the endotoxin content of E-2 is NMT 5 EU/mg.

In the next step, the CEX Elution fractions based on analytical results of charge variant on E-2 were pooled and diluted using AEX Equilibration buffer (EB-4, Tris: 20 mM, pH 8.0) followed by filtration using 0.2 μm filter. In the next step, the anion exchange column (Capto Q, GE Healthcare) was equilibrated with 10 CVs of Equilibration Buffer-4 (EB-4, Tris: 20 mM, pH 8.0) at a flow rate of 250 cm/h. The sample was loaded on to column at a flow rate of 250 cm/h which was followed by 2 CVs of the wash buffer-3 (WB-3, EB-4, Tris: 20mM, pH 8.0) at the flow rate of 250 cm/h. The column was regenerated with 5CVs of regeneration buffer (Tris: 20 mM, NaCl: 1M, pH 8.0) and flow through (FT) was collected for further analysis referred as “AEX FT”. It was analysed for protein quantity, Glycans, HCP, charge variants, aggregates and endotoxin content. At this step, the endotoxin content is not more than (NMT) 0.5 EU/mg and aggregate content is not more than 3%. Further, “AEX FT” was filtered using Prefilter and Nano filter connected in series, at a differential pressure of 1.5±0.5 Bar and Nanofiltrate was concentrated and diafiltered with Diafiltration buffer (PB: 51 mM, Trehalose dihydrate: 20 mg/ml, pH 6.0±0.2). The diafiltered sample was spiked with Trehalose di-hydrate to make up the final conc. up to 60 mg/ml and with 5% Polysorbate 20 to make up the final conc. up to 0.04% and further diluted to achieve the specified bevacizumab concentration to produce bevacizumab drug substance (DS). The DS was subjected to comprehensive analytical characterization which includes but not limited to Protein quantity, Glycans, HCP, charge variants, aggregates, endotoxin content, particulate matter, and invitro and invivo receptor binding assays. On DS, the endotoxin content is less than 0.16 EU/mg, aggregate content is 0.8% and the sub-visible particulate matter limits as below, lies within the limits specified by the present invention.
(i) ≥10 μm particles is not more than 50 particles/ml.
(ii) ≥25 μm particles is not more than 5 particles/ml.
(iii) ≥50 μm particles is not more than 2 particles/ml.

The ability to achieve the limits of the present invention with respect to endotoxin, Aggregate reduction and HCP reduction is provided here below at Table 1.

TABLE 1
Effectiveness of the process of the present invention

| | Endotoxin | Aggregate | HCP level |
|---|---|---|---|
| | level after | level after | after |
| Process Steps | each step | each step | each step |
| SPTFF | NMT 10 EU/mg | NA | NA |
| Protein A | NMT 10 EU/mg | NMT 5% | NMT 5000 ppm |
| Chromatography | | | |
| Cation Exchange | NMT 5 EU/mg | NMT 5% | NMT 200 ppm |
| chromatography | | | |
| Anion Exchange | Less Than 0.5 EU/mg | Less | NMT 100 ppm |
| Chromatography | | Than 3% | |
| Bevacizumab DS | Less Than 0.16 | 0.8% | 1 ppm |
| | EU/mg | | |

### Example 2: Composition According to the Present Invention

The bevacizumab as obtained in example 1. The composition of the present invention may be formulated using the various ingredients as below as disclosed in Table 2.

TABLE 2
Illustrative ophthalmic composition of the present invention

| | C1 | C2 | C3 |
|---|---|---|---|
| Bevacizumab | Bevacizumab | Bevacizumab | Bevacizumab |
| Sugar | Sucrose | Trehalose | Trehalose |
| Buffer | Acetate buffer | Histidine buffer | Phosphate buffer |
| Surfactant | Polysorbate 20 | Polysorbate 20 | Polysorbate 20 |

The Composition 3 (C3), was proceeded for further experimentation as below. Some illustrative examples are at Table 3.

TABLE 3
Ophthalmic composition of the present invention

| | | |
|---|---|---|
| C31 | C32 | C33 |

| | | | | | | |
|---|---|---|---|---|---|---|
| Bevacizumab | 25 | mg/mL | 25 | mg/mL | 25 | mg/mL |
| Trehalose | 50 | mg/mL | 70 | mg/mL | 60 | mg/mL |
| Phosphate buffer | 55 | mM | 45 | mM | 51 | mM |

| | | | |
|---|---|---|---|
| Polysorbate | 0.02% | 0.03% | 0.04% |
| pH | 6.2 | 6.2 | 6.2 |

The composition C33 of the present invention was analyzed further here below and the results are presented in the following examples.

### Example 3: Analytical Characterization of the Composition of the Present Invention in Prefilled Syringe

Single use pre-filled syringe was used to fill the ophthalmic composition of bevacizumab, which is present as a solution. Three batches of bevacizumab in PFS were extensively characterized to determine physicochemical properties, biological activity, immunochemical properties, purity and impurities by appropriate techniques. The product characteristics comply with the acceptance criteria as shown in Table 4 below:

TABLE 4
Analytical Characterization of bevacizumab in PFS

| | | | Mean of 3 | |
|---|---|---|---|---|
| No. | Test | Acceptance Criteria of DP | Batches | SD |
| 1. | pH | 6.0-6.4 | 6.13 | 0.06 |
| 2. | Protein concentration | 25 ± 0.5 mg/mL | 25.17 | 0.12 |
| | (mg/mL) | | | |
| 3. | Peptide Map | The profile of the peptide map | Complies | — |
| | | of the sample solution is | | |
| | | qualitatively similar to the | | |
| | | reference standard solution | | |
| 4. | Aggregation (%) | NMT 2% | 0.85 | 0.24 |
| 5. | Glycan content (%) | Sum of all oligosaccharides | 84.13 | 2.64 |
| | | without galactose: NLT 70.0% | | |
| 6. | Charge variants (%) | Acidic NMT 45% | 14.43 | 2.26 |
| | | Basic NMT 5% | 0.83 | 0.12 |
| 7. | pI | The pI of the main peak | Complies | — |
| | | obtained from the sample | | |
| | | solution differs by NMT ± 0.2 | | |
| | | pI units from the pI of the | | |
| | | corresponding peak obtained | | |
| | | from the Standard solution | | |
| 8. | Low molecular weight | NMT 5% | 2.03 | 0.32 |
| | impurities (%) | | | |
| 9. | Non-glycosylated heavy | NMT 2.0% | 0.77 | 0.12 |
| | chain impurity (%) | | | |
| 10. | Binding potency (%) | 80%-120% of the Stated | 103.83 | 6.85 |
| | | potency | | |
| 11. | Neutralization potency (%) | NLT 80.0% | 103.67 | 6.11 |
| | | and NMT 120.0% of the | | |
| | | stated potency | | |
| 12. | Sub visible Particles ≥10 μm | Not More Than 50/mL | 22 | 7.64 |
| 13. | Sub visible Particles ≥25 μm | Not More Than 5/mL | 1 | 0 |

### Example 4: Stability Study of the Composition of the Present Invention in Single Use PFS

Bevacizumab (ophthalmic grade) in single use PFS was subjected for long term stability study at 5° C.±3° C. for 24 months. The PFS were filled with 25 mg/ml bevacizumab, phosphate buffer, Polysorbate 20, and Trehalose dihydrate, pH 6.2. During the complete stability program, no significant changes in protein concentration and pH was detected. Data of 24 months stability studies of drug product extensively analyzed for physicochemical properties, biological activity, immunochemical properties, purity and impurities and showing that the product is stable at 5° C.±3° C. for 24 months (Table 5).

TABLE 5
Long term stability study of ophthalmic grade bevacizumab at 5° C. ± 3° C. for
24 months

| | Specification of | | 3 | 6 | 9 | 12 | 24 |
|---|---|---|---|---|---|---|---|
| Test | End of Shelf Life | Initial | months | months | months | months | months |
| Appearance | Clear colorless to | complies | complies | complies | complies | complies | complies |
| | pale brown liquid, | | | | | | |
| | free of visible | | | | | | |
| | particles | | | | | | |
| pH | 6.0-6.4 | 6.10 | 6.23 | 6.03 | 6.11 | 6.18 | 6.35 |
| Protein | 25.5 ± 0.5 mg/mL | 25.10 | 25.07 | 25.23 | 25.15 | 25.20 | 25.37 |
| conc. | | | | | | | |
| (mg/mL) | | | | | | | |
| SDS PAGE | Major bands | complies | complies | complies | complies | complies | complies |
| | should be | | | | | | |
| | observed at | | | | | | |
| | 150 kD in non | | | | | | |
| | reduced condition; | | | | | | |
| | at 50 kD, and | | | | | | |
| | 25 kD in reduced | | | | | | |
| | condition | | | | | | |
| % Binding | 80%-125% of the | 100.6 | 95.9 | 99.7 | 111.8 | 112.5 | 100.7 |
| efficiency | Stated potency | | | | | | |
| (ELISA) | | | | | | | |
| VEGF | NLT 80.0% and | 108.1 | 105.4 | 104.1 | 97.3 | 108.7 | 93.6 |
| Neutralization | NMT 125.0% of | | | | | | |
| potency | the stated potency | | | | | | |
| % NGHC | NMT 2.0 % | 0.70 | 0.73 | 0.78 | 0.70 | 0.70 | 0.61 |
| impurity | | | | | | | |
| % | NMT 3.0% | 0.81 | 1.32 | 1.47 | 1.93 | 2.01 | 2.64 |
| Aggregation | | | | | | | |
| % | Sum of all | 85.90 | 85.49 | 80.08 | 85.87 | 83.32 | 83.81 |
| Oligosaccharides | oligosaccharides | | | | | | |
| without | without galactose: | | | | | | |
| galactose | NLT 70.0% | | | | | | |
| Particles | Not More Than | 46 | 44 | 23 | 16 | 11 | 8 |
| ≥10μ | 50/mL | | | | | | |
| Particles | Not More Than | 1 | 1 | 1 | 1 | 1 | 1 |
| ≥25μ | 5/mL | | | | | | |

The product characteristics complies with the acceptance criteria for the entire duration showing that the product is stable at 5° C.±3° C. for 24 months.

In prior art, repackaged bevacizumab from vial (Avastin®) into syringes is not found to be stable at longer duration. There are reports in which the quality of compounded bevacizumab repackaged into plastic syringes (Polypropylene and polycarbonate) for intravitreal injection has been analysed up to 3 or 6 months. In these studies, changes in the immunoglobulin G (IgG) content, the presence of silicone oil micro droplets, increase in sub-visible particles and protein aggregation were reported. It was also observed that repackaged bevacizumab from different suppliers showed varying product quality. In one of the prior art 15.9% degradation of anti-VEGF activity was observed in a 6 months stability time period of compounded bevacizumab.

Due to stability related issues USFDA draft guidance proposing to limit expiration of pre-filled Avastin® PFS to 5 days. In India, Drug Controller General of India (DCGI) drafted a guideline for compounding procedure and it was mentioned that the use of these syringes should not be done beyond 14 days. Hence, stability of bevacizumab for long duration with desired qualities in repackaged syringes is a critical issue and responsible for eye inflammation. The current invention overcome these limitations and provides a long storage of bevacizumab by meeting all the acceptance criteria set herein during the shelf life.

In comparison with prior art literature, the composition of the present invention, possesses a particle size of >10 μm and >25 μm during entire shelf life of 2 years. The ophthalmic composition of the present invention is stable at a temperature of 2 to 8° C. for at least two years and is safe, non-toxic and efficacious for administration during the shelf life.

### Example 5: Toxicity Study of the Composition of the Present Invention

Single use pre-filled syringes filled with ophthalmic grade bevacizumab tested for intravitreal repeated dose (4-week) toxicity study with 4-week recovery period in rabbit eyes. The study was conducted in GLP facility to assess the toxicity of bevacizumab in eye. The PFS were filled with 25 mg/ml bevacizumab, 51 mM PB, 0.04% Polysorbate 20, and 60 mg/ml Trehalose dihydrate, pH 6.2. The product complied with all the parameters of ophthalmic composition during the shelf life.

During its therapeutic use in ocular diseases, Bevacizumab is directly injected into the vitreous humour, which comprises a special compartment of the eye. The test product Bevacizumab, or the vehicle (diluent) control sample, were administered by intravitreal injections into both eyes of each rabbit, once in two weeks for four weeks, i.e. on days 1, 15 and 29, during the period of the study.

The rabbits were observed for the incidence of mortality and signs of local and systemic toxicity during the study and then were sacrificed and subjected to a complete necropsy. Additional concurrent recovery groups of three rabbits of each sex at vehicle control and high dose level were treated similarly but, after cessation of treatment period, were further observed for reversal of toxicity/delayed toxicity, if any, for a period of 28 days.

The study provided information on target organs, the possibilities of cumulation, reversibility of toxic effects, an estimate of a no-effect level of exposure, which was used for establishing safety criteria for human exposure. Three levels of doses had been selected in toxicity studies. The dose level used in the study was a multiple of the human equivalent dose (HED) of Bevacizumab for rabbit i.e. 1.25 mg, 2.50 mg or 3.75 mg per eye which are respectively 1×, 2× and 3× of the absolute therapeutic human dose and was injected in a volume of 50 μL.

Based on the findings of the study, it was concluded that, the product did not induce any systemic effects, adverse or otherwise, in rabbits treated at and up to the dose level of 3.75 mg per eye. Ophthalmic grade bevacizumab was well tolerated in the eyes of rabbits following its intravitreal injections at the dose levels of 1.25 mg, 2.50 mg or 3.75 mg per eye; and No observed adverse effect level (NOAEL) of the ophthalmic composition of the present invention in rabbit was found to be greater than 3.75 mg per eye. Hence, in intravitreal preclinical studies it is found that the drug is safe for intravitreal route.

Specifically, the invention provides pharmaceutical compositions with significantly reduced endotoxin level and particulate matter levels that are suitable for intravitreal use. The invention also relates to methods of reducing the level of PMT and endotoxins within certain compositions, such as pharmaceutical compositions, that can be used for intraocular delivery.

### Example 6: Comparison of the Composition of the Present Invention with Prior Art Composition

The composition of the present invention prepared as set out in the preceding examples was compared with current compositions available in the market and that are being administered to the patients.

### A. Comparison of Sub Visible Particulate Matter

The sub visible particulate matter was tested as per USP 789 method by Light Obscuration Particulate Count (LOPC). Appropriate volume particle free glass bottles were used for the test. Samples from 10 PFS which makes around 1 ml volume was withdrawn in one glass bottle. Further, the sample was diluted with particle free water to obtain final 25 ml volume. The contents of the sample were mixed slowly inverting the container 20 times successively without introducing air bubbles while pooling total containers in particle free container. The sample containers were allowed to stand for at least 15 minutes prior analysis to remove air bubbles.

Four portions of not less than 5 ml each were removed and the number of particles equal to and greater than 10 μm, 25 μm and 50 μm were counted. The results are presented after disregarding the result obtained in first portion.

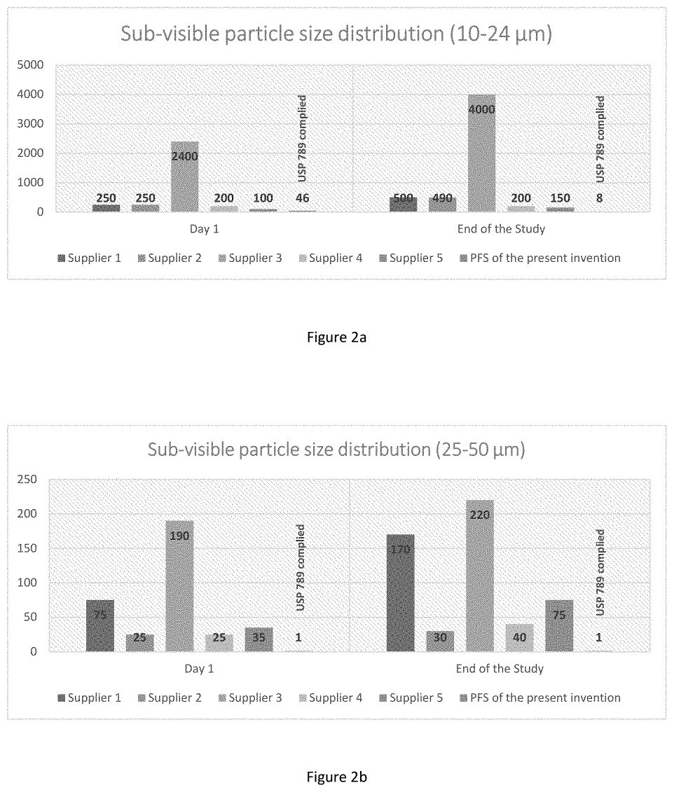

The sub-visible particulate matter of repackaged bevacizumab from five UK compounding pharmacies (S1-S5) and compared with the ophthalmic composition of the present invention. (Eye (2013) 27,1090-1097). The results are presented as FIGS. 2a and 2b. From the FIGS. 2a and 2b, it can be clearly seen that the particle size of the present invention is within the range as set out in the present invention, thereby the potential for incidences of eye inflammation are very low.

### B. Comparison of Storage Stability of the Composition of the Present Invention with that of Prior Art

The storage stability of repackaged bevacizumab is compared with the ophthalmic composition of the present invention and that of reconstituted bevacizumab and the results are presented in Table 6 below.

TABLE 6
Comparison of storage stability of the prior art
composition with that of the present invention

| S. | | Stability Period | |
|---|---|---|---|
| No. | Research Article | in the study | |
| 1 | Quality of bevacizumab compounded for | 14 | days |
| | intravitreal administration. | | |
| | Eye. 2013; 27(9): 1090-7. | | |
| 2 | Storage stability of bevacizumab in | 6 | months |
| | polycarbonate and polypropylene syringes. | | |
| | Eye. 2015; 29(6): 820-7. | | |
| 3 | Silicone Oil Microdroplets and Protein | 3 | months |
| | Aggregates in Repackaged Bevacizumab and | | |
| | Ranibizumab: Effects of Long-term Storage | | |
| | and Product Mishandling. | | |
| | Invest Ophthal mol Vis Sci. 2011; 52(2): | | |
| | 1023-1034. | | |
| 4 | Long-term stability of bevacizumab repackaged | 3 | months |
| | in 1 mL polypropylene syringes for | | |
| | intravitreal administration. | | |
| | Retina. 2010; 30(6): 887-92. | | |
| 5 | Quantification, Microbial Contamination, | 3 | months |
| | Physico-chemical Stability of Repackaged | | |
| | Bevacizumab Stored Under Different Conditions. | | |
| | Curr Pharm Biotechnol. 2014; 15(2): 113-9. | | |
| 6 | Six-month stability of bevacizumab (AVASTIN) | 6 | months |
| | binding to Vascular endothelial growth factor | | |
| | after withdrawl into a syringe and refrigeration | | |
| | or freezing. | | |
| | Retina. 2006; 26(5): 519-22. | | |
| 7 | Ophthalmic composition of the present invention | 24 | months |

In the present invention, the composition of the present invention is found to be stable for two years, which is significantly different from the compositions of prior art as seen above, rendering economic advantage and transportation advantage.

### C. Comparison of Bevacizumab Concentration of the Present Invention with Prior Art Composition

In a study, “Retina2006; 26(5):519-22”, degradation of bevacizumab was shown that was withdrawn into plastic tuberculin syringe which was commonly used for compounding of drug in most of the countries. The drug was stored for six months at 4° C. The degradation pattern was seen as mentioned below in Table 7 below:

TABLE 7
Stability of the composition of prior art composition

| | | Mean Bevacizumab | |
|---|---|---|---|
| Stability Time point of | | Concentration | Degradation Of |
| compounded Bevacizumab | | (mg/mL) | Bevacizumab (%) |
| 0 | day | 25.1 | 0.0 |
| 1 | week | 24.7 | 1.6 |
| 1 | month | 23.1 | 8.0 |
| 3 | month | 22.9 | 8.8 |
| 6 | month | 21.1 | 15.9 |

At 6 months 15.9% of degradation was observed in compounded bevacizumab of prior art.

In the present invention, no degradation of protein has been observed in two years of time period which is also evidenced by the % binding efficiency (ELISA) and % VEGF Neutralization potency assay during the two years of stability period (See table 8).

TABLE 8
Composition of the present invention

| | | | 3 | 6 | 9 | 12 | 18 | 24 |
|---|---|---|---|---|---|---|---|---|
| Test | Specification | Initial | months | months | months | months | months | months |
| *Mean % | NLT 80.0% | 106.05 | 96.3 | 102.75 | 106.75 | 106.55 | 104.15 | 99.75 |
| Binding | and NMT | | | | | | | |
| efficiency | 125.0% of | | | | | | | |
| (ELISA) | the stated | | | | | | | |
| | potency | | | | | | | |
| *Mean VEGF | 80%-125% | 107.15 | 106.2 | 97.9 | 100.9 | 104.45 | 101.85 | 95.55 |
| Neutralization | of the | | | | | | | |
| potency % | Stated | | | | | | | |
| | potency | | | | | | | |

*Mean is calculated from two stability batches of PFS containing ophthalmic grade bevacizumab.

Therefore, the present invention as presented in the composition as disclosed herein, formulated with the bevacizumab obtained by the process as presented herein and in the prefilled syringe as disclosed herein, alone possesses the desired product characteristics, stability and the efficacy.

The composition, the process for preparing the composition and the device comprising the composition such as prefilled syringe is achieved after considerable human efforts and experimentation and has its inherent merits.

1. An ophthalmic composition of Bevacizumab;
a. wherein the limits of Bacterial Endotoxin Test (BET) in the range of 0.001 to 0.4 EU/mg and preferably in the range of 0.001 to 0.2 EU/mg and more preferably in the range of 0.001 to 0.16 EU/mg;
b. wherein the particulate matter is;
iv. 1 to 50 particles ≥10 μm in diameter per ml,
v. 0 to 5 particles ≥25 μm in diameter per ml, and,
vi. 0 to 2 particles ≥50 μm in diameter per mL; and;
c. wherein the composition has an aggregate of 0.1 to 5%, more preferably 0.1 to 4% and most preferably 0.1 to 3.5% for a period of 2 to 3 years, preferably 2 to 2.5 years, most preferably 2 years.

2. A process of preparing the ophthalmic composition as claimed in claim 1 comprising the steps of:
i. culturing the cells of bevacizumab by continuous fermentation method in CHO cells;
ii. subjecting cell free harvest obtained from CHO cell culture to adsorption-based depth filtration for further clarification;
iii. concentrating the sample of step (ii) using single pass tangential flow filtration to obtain the concentrated harvest;
iv. subjecting the concentrated harvest of step (iii) to protein A chromatography to capture bevacizumab and obtaining an eluate containing partially purified bevacizumab;
v. subjecting eluate of step (iv) to low pH virus inactivation to obtain the viral inactivated sample;
vi. subjecting the viral inactivated sample of step (v) to a further Cation exchange chromatography for additional purification to obtain an eluate containing primarily highly purified bevacizumab;
vii. subjecting the eluate of step (vi) to anion exchange chromatography to remove the trace impurities e.g. Host Cell Proteins (HCP), Host Cell DNA (HCD), endotoxins;
viii. subjecting the eluate of step (vii) to virus reduction filtration and complete removal of viruses;
ix. concentrating and diafiltrating the sample of step (viii) to obtain bevacizumab; and;
x. formulating the sample obtained from step (ix) to achieve the composition of bevacizumab of the present invention.

3. The process as claimed in claim 2, wherein the low virus inactivation is carried out at a pH in the range of pH 3.0 to 5.0 preferably in the range of 3.0 to 4.0, most preferably in the range of pH 3.5 to 4.0.

4. The process as claimed in claim 2, wherein the stationary phase of the cation exchange chromatography is selected from the group comprising SO₃⁻Sulfoisobutyl, SO₃⁻Sulfoethyl, Sulfopropyl, Carboxymethyl, preferably Sulfonate and the stationary phase of the anion exchange chromatogrpahy is selected from the group comprising Diethylaminoethyl, Quarternaryamine, Polyquaternium, N-benzyl-N-methyl ethanol amine, preferably Quarternaryamine and diafiltration is conducted in the presence TFF-II Diafiltration Buffer.

5. The ophthalmic composition produced by the process as claimed in claim 2,
a. wherein the limits of Bacterial Endotoxin Test (BET) in the range of 0.001 to 0.4 EU/mg and preferably in the range of 0.001 to 0.2 EU/mg and more preferably in the range of 0.001 to 0.16 EU/mg;
b. wherein the particulate matter is;
i. 1 to 50 particles ≥10 μm in diameter per ml,
ii. 0 to 5 particles ≥25 μm in diameter per ml, and,
iii. 0 to2 particles ≥50 μm in diameter per mL;
c. wherein the composition has an aggregate of 0.1 to 5%, more preferably 0.1 to 4% and most preferably 0.1 to 3.5% for a period of 2 to 3 years, preferably 2 to 2.5 years, most preferably 2 years.

6. The ophthalmic composition as claimed in claim 1, wherein the composition comprises bevacizumab, a buffer, a stabilizer and a surfactant.

7. The ophthalmic composition as claimed in claim 6, wherein the concentration of bevacizumab in the composition is in the range of 24 mg/ml to 26 mg/ml, preferably in the range of 25 mg/ml to 26 mg/ml, more preferably 25 mg/ml.

8. The ophthalmic composition as claimed in claim 6, wherein the buffer is selected from the group comprising phosphate, citrate, acetate, histidine, succinate, gluconate, glycine, more preferably phosphate buffer and is the concentration of 40 mM to 60 mM, more preferably in the range of 50 mM to 60 mM and the pH is in the range of 6.0 to 7.0, more preferably 6.1 to 6.3.

9. The ophthalmic composition as claimed in claim 6, wherein the stabilizer is a saccharide selected from monosaccharide, disaccharide, trisaccharide, polysaccharide, sugar alcohol, reducing sugar, nonreducing sugar, preferably the saccharide is a glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose more preferably trehalose, and is in the range of 40 mg/mL to 70 mg/mL, preferably the range may be 45 mg/mL to 65 mg/mL, more preferably in the range of 50 mg/mL to 60 mg/mL.

10. The ophthalmic composition as claimed in claim 6, wherein the surfactant is selected from the group comprising a nonionic surfactant, preferably polysorbate polysorbate 20 or polysorbate 80, more preferably polysorbate 20 and is in the range of 0.2 mg/mL to 0.6 mg/mL, preferably in the range of 0.3 mg/mL to 0.5 mg/mL, more preferably in the range of 0.35 mg/mL to 0.45 mg/mL.

11. The ophthalmic composition as claimed in claim 1, administered as predetermined dose be in the form of a vial, a cartridge or a pen or a prefilled syringe, preferably a pre-filled syringe, preferably single use pre-filled syringe, with a fill volume of 140 microL to 200 microL and with a dose of 50 microL.

12. The prefilled syringe as claimed in claim 11, wherein the injection is administered intravitreally.

13. The ophthalmic composition and prefilled syringe as claimed in claim 1, for its use in ocular disorders selected from the group comprising wet Age related macular degeneration, choroidal neovascularization, retinal angiomatous proliferation, pathologic myopia, angioid streaks, Best disease, Adult vitelliform dystrophy, Central serous chorioretinopathy, Punctate inner choriodopathy, Multifocal choroiditis, Presumed ocular histoplasmosis syndrome, Choroidal osteoma, Toxoplasmosis, Uveitis, Pseudotumor cerebri, Peripapillary Idiopathic Retinal neovascularization, Proliferative diabetic retinopathy, Sickle cell retinopathy, Retinopathy of prematurity, Eales disease, Macular edema, Diabetic retinopathy, Central retinal vein occlusion, Branch retinal vein occlusion, Pseudophakic, Uveitic, Occlusive vasculitis, Retinitis pigmentosa, Neovascular glaucoma, Central retinal vein occlusion, Branch retinal vein occlusion, Proliferative diabetic retinopathy, Central retinal artery occlusion, Ocular ischemic syndrome, Radiation induced, Radiation optic neuropathy, Radiation retinopathy, Breast cancer with choroidal metastasis, Melanoma associated neovascularization, Macroaneurysm, Vasoproliferative tumor, Coats disease, Juxtapapillary capillary hemangioma, Idiopathic macular telangiectasis, Polypoidal choroidal vasculopathy, Central serous chorioretinopathy, Nonarteritic anterior ischemic optic neuropathy, Herpetic corneal neovascularization, Cicatricial pemphigoid corneal neovascularization, Posterior capsular neovascularization, Corneal graft rejection neovascularization, Dry eye associated corneal neovascularization, Bleb revision, Adjunct to glaucoma filtering surgery, preferably wet age-related macular degeneration.

14. A kit comprising the ophthalmic composition in a prefilled syringe as claimed in claim 1, comprising a prefilled syringe, a needle with gauge in the range of 29 to 34 gauge, instructions for use in a blister pack.

15. The ophthalmic composition and prefilled syringe as claimed in claim 5, for its use in ocular disorders selected from the group comprising wet Age related macular degeneration, choroidal neovascularization, retinal angiomatous proliferation, pathologic myopia, angioid streaks, Best disease, Adult vitelliform dystrophy, Central serous chorioretinopathy, Punctate inner choriodopathy, Multifocal choroiditis, Presumed ocular histoplasmosis syndrome, Choroidal osteoma, Toxoplasmosis, Uveitis, Pseudotumor cerebri, Peripapillary Idiopathic Retinal neovascularization, Proliferative diabetic retinopathy, Sickle cell retinopathy, Retinopathy of prematurity, Eales disease, Macular edema, Diabetic retinopathy, Central retinal vein occlusion, Branch retinal vein occlusion, Pseudophakic, Uveitic, Occlusive vasculitis, Retinitis pigmentosa, Neovascular glaucoma, Central retinal vein occlusion, Branch retinal vein occlusion, Proliferative diabetic retinopathy, Central retinal artery occlusion, Ocular ischemic syndrome, Radiation induced, Radiation optic neuropathy, Radiation retinopathy, Breast cancer with choroidal metastasis, Melanoma associated neovascularization, Macroaneurysm, Vasoproliferative tumor, Coats disease, Juxtapapillary capillary hemangioma, Idiopathic macular telangiectasis, Polypoidal choroidal vasculopathy, Central serous chorioretinopathy, Nonarteritic anterior ischemic optic neuropathy, Herpetic corneal neovascularization, Cicatricial pemphigoid corneal neovascularization, Posterior capsular neovascularization, Corneal graft rejection neovascularization, Dry eye associated corneal neovascularization, Bleb revision, Adjunct to glaucoma filtering surgery, preferably wet age-related macular degeneration.

16. The ophthalmic composition and prefilled syringe as claimed in claim 12, for its use in ocular disorders selected from the group comprising wet Age related macular degeneration, choroidal neovascularization, retinal angiomatous proliferation, pathologic myopia, angioid streaks, Best disease, Adult vitelliform dystrophy, Central serous chorioretinopathy, Punctate inner choriodopathy, Multifocal choroiditis, Presumed ocular histoplasmosis syndrome, Choroidal osteoma, Toxoplasmosis, Uveitis, Pseudotumor cerebri, Peripapillary Idiopathic Retinal neovascularization, Proliferative diabetic retinopathy, Sickle cell retinopathy, Retinopathy of prematurity, Eales disease, Macular edema, Diabetic retinopathy, Central retinal vein occlusion, Branch retinal vein occlusion, Pseudophakic, Uveitic, Occlusive vasculitis, Retinitis pigmentosa, Neovascular glaucoma, Central retinal vein occlusion, Branch retinal vein occlusion, Proliferative diabetic retinopathy, Central retinal artery occlusion, Ocular ischemic syndrome, Radiation induced, Radiation optic neuropathy, Radiation retinopathy, Breast cancer with choroidal metastasis, Melanoma associated neovascularization, Macroaneurysm, Vasoproliferative tumor, Coats disease, Juxtapapillary capillary hemangioma, Idiopathic macular telangiectasis, Polypoidal choroidal vasculopathy, Central serous chorioretinopathy, Nonarteritic anterior ischemic optic neuropathy, Herpetic corneal neovascularization, Cicatricial pemphigoid corneal neovascularization, Posterior capsular neovascularization, Corneal graft rejection neovascularization, Dry eye associated corneal neovascularization, Bleb revision, Adjunct to glaucoma filtering surgery, preferably wet age-related macular degeneration.

17. A kit comprising the ophthalmic composition in a prefilled syringe as claimed in claim 5, comprising a prefilled syringe, a needle with gauge in the range of 29 to 34 gauge, instructions for use in a blister pack.

18. A kit comprising the ophthalmic composition in a prefilled syringe as claimed in claim 13, comprising a prefilled syringe, a needle with gauge in the range of 29 to 34 gauge, instructions for use in a blister pack.
